SEQUENCE LISTING (8 sequences)

SEQ ID NO: 1 - DNA, 24 bp, Artificial Sequence
  PCR Primer
ataagaattctgcaacaactgctg

SEQ ID NO: 2 - DNA, 25 bp, Artificial Sequence
  PCR Primer
ctccatgtttttctaggtctcgaga

SEQ ID NO: 3 - DNA, 28 bp, Artificial Sequence
  PCR Primer
caccatgagagtgaaggggatacagaag

SEQ ID NO: 4 - DNA, 27 bp, Artificial Sequence
  PCR Primer
cctcccattttatagcaaagctctttc

SEQ ID NO: 5 - DNA, 42 bp, Artificial Sequence
  PCR Primer
gcggcccagccggccavagtgawggrgawacagargaattgg

SEQ ID NO: 6 - DNA, 38 bp, Artificial Sequence
  PCR Primer
ggctcgagatcttttttctctcyscaccactcttctcy

SEQ ID NO: 7 - DNA, 26 bp, Artificial Sequence
  PCR Primer
gggacgcgtatgavagtgawggrgaw

SEQ ID NO: 8 - DNA, 33 bp, Artificial Sequence
  PCR Primer
aaatctagatttgacmayttgcchcccatytta

(19) United States
(12) Patent Application Publication
(10) Pub. No.: US 2008/0220528 A1
(43) Pub. Date: Sep. 11, 2008

(54) HIV GENE CLONING STRATEGY

(75) Inventors: John A. Howe, Green Brook, NJ (US); Robert O. Ralston, Union, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: SCHERING CORPORATION

(21) Appl. No.: 11/963,007

(22) Filed: Dec. 21, 2007

Related U.S. Application Data
(60) Provisional application No. 60/876,605, filed on Dec. 22, 2006.

(51) Int. Cl. C12N 15/74; C12N 1/20; C12N 15/11

(52) U.S. Cl. 435/476; 435/252.3; 435/252.33; 536/23.72

(57) ABSTRACT

The invention relates to methods for cloning Human Immunodeficiency Virus (HIV) genes, in particular HIV envelope genes. The invention also relates to cloning strategies for mapping resistance determinants for HIV genes, in particular HIV envelope genes.

### REFERENCE TO CROSS RELATED APPLICATIONS

This application claims the benefit of priority under 35 USC 119(e) of provisional patent application U.S. Ser. No. 60/876,605 filed Dec. 22, 2006, the disclosure of which is hereby incorporated by reference in its entirety.

### 1. FIELD OF THE INVENTION

The field of the invention relates to cloning strategies for Human Immunodeficiency Virus (HIV) genes, in particular HIV envelope genes and cloning strategies for mapping resistance determinants for HIV genes, in particular HIV envelope genes.

### 2. BACKGROUND OF THE INVENTION

Human immunodeficiency virus (HIV), the causative agent of acquired immunodeficiency syndrome (AIDS), replicates in and destroys the CD4+ lymphocytes of infected individuals. HIV particles gain entry into CD4+ cells through a membrane attachment and fusion process that is mediated by envelope protein spikes on the surface of the HIV-1 particle, HIV envelope spikes are composed of two subunits, the surface glycoprotein 120 (gp120) and the transmembrane glycoprotein subunit gp41. The gp120 and gp41 subunits are produced by proteolytic cleavage of the gp160 kDA polyprotein precursor that is encoded by a single viral open reading frame. Viral attachment begins with binding of gp120 to the CD4 T cell receptor followed by a conformational rearrangement of gp120 that facilitates its binding to either the CCR5 or CXCR4 chemokine coreceptors. After co-receptor binding further gp120 conformational changes activate the fusion properties of gp41 leading to insertion of the gp41 fusogenic peptide into the cellular membrane and subsequent membrane fusion.

Viral entry inhibitors are an emerging class of HIV therapies that target steps in the entry process. Currently, this class of drugs is represented by two members, enfuvirtide which interacts with gp41 and prevents fusion of the viral and cellular membranes, and maraviroc, a small molecule antagonist of the CCR5 receptor that inhibits gp120 bonding. A number of other antiviral agents are in the process of clinical and pre-clinical development. These include antibody, peptide and small molecules that block gp120 engagement of CD-4 binding or the coreceptors CCR5 and CXCR4. Since HIV-1 has a great propensity to mutate under selective pressure, the development of drug resistance remains an issue. Thus, resistance to novel entry inhibitors is expected and must be characterized for successful clinical development of these agents. In vitro and in vivo studies have shown that Enfuvirtide has a low genetic barrier for resistance (Briz et al., 2006 J. Antimicrob Chemther 57(4):619-27) with various amino acid changes in the HR1 domain of gp41 reducing susceptibility to this drug. Resistance to CCR5 and CXCR4 antagonists can occur as a result of a shift in co-receptor usage or from mutations in the HIM envelope genomic regions which allow gp120 to interact with the co-receptor in the presence of drug (Westby et al, 2006 J. Virol. 80 (10), 4090-20 Maroszan et al., 2005 Virology 338 (1), 182-99).

Characterization of resistance to entry inhibitors requires the cloning and DNA sequence analysis of gp160 sequences from resistant strains generated in laboratory studies or in clinical tests of novel entry inhibitors. Because prediction of phenotype from genotype is not currently possible for gp120, functional assays are required to characterize resistance mutants that develop after treatment with CCR5 or CXCR4 inhibitors. This functional evaluation requires that envelope gene coding sequences from resistant strains need to be cloned into appropriate vectors for expression in mammalian cells so that functional assays for co-receptor usage or drug susceptibility can be performed.

Cloning of the HIM envelope gene gp160 from infected cell DNA or from clinical plasma samples is usually carried out in a two step process. In the first step nested primer polymerase chain reactions (PCR) are used to obtain a DNA copy of the viral gene. The PCR primers used for amplification are engineered to contain exogenous restriction enzyme recognition sequences to facilitate cloning of the amplified gp160 gene into bacterial vectors. In theory, PCR products containing full-length gp160 can be cleaved with restriction enzymes that cut the engineered recognition sequences, and then ligated into bacterial vectors that have been rendered receptive by cleavage with cognate enzymes. However, although this protocol is a standard procedure for cloning most PCR products, inserts containing full-length, in-frame gp160 are extremely difficult to obtain. PCR based methods for isolating HIV gp160 genes involve the ligation of envelope gene containing PCR fragments into vector plasmid to generate open circle plasmids that are subsequently transformed into competent bacterial cells for amplification. This strategy also has a poor success rate. Accordingly, a more reliable and efficient method of cloning HIV envelope protein is needed.

### 3. SUMMARY OF THE INVENTION

The invention relates to cloning strategies for Human Immunodeficiency Virus (HIV) genes, in particular HIV envelope genes. In one embodiment, the invention relates to a method of cloning an HIV gene comprising transforming a host cell with a first linearized polynucleotide comprising at least one regulatory element and encoding at least one selectable marker and, optionally, all or part of a polypeptide of interest and a second linearized polynucleotide encoding all or part of a polypeptide of interest, wherein the first polynucleotide and the second polynucleotide each comprise homologous sequences with respect to each other, sufficient to allow for homologous recombination between the first polynucleotide and the second polynucleotide.

In one embodiment the linearized first polynucleotide is a vector and the regulatory element is a mammalian regulatory element and the linearized second polynucleotide encodes all or part an HIV protein and the host cell is a bacterial cell. In another embodiment the linearized first polynucleotide encodes all or part of an HIV protein.

This invention also relates to the host cells comprising the first polynucleotide and the second polynucleotide sequence.

This invention further relates to the polynucleotide sequence which is the product of the homologous recombination of the first polynucleotide with the second polynucleotide (e.g., supercoiled DNA).

The invention also relates to chimeric envelope sequences produced by the homologous recombination of a first polynucleotide with the second polynucleotide.

This invention also relates to kits comprising a linearized first polynucleotide and a linearized second polynucleotide, wherein the linearized second polynucleotide encodes all or part an HIV protein.

This invention further relates to methods of mapping resistance to an HIV therapeutic (e.g., viral entry inhibitors) using the chimeric envelope sequences produced by the homologous recombination of the first polynucleotide with the second polynucleotide.

### BRIEF DESCRIPTION OF THE DRAWINGS

FIG. 1: pSV7-ADA-gp160 mammalian expression vector. DNA sequences encoding for ADA gp120 and gp41, and recognition sites for the Nde I and Eco NI restriction enzymes which cleave in the gp120 DNA coding sequence are shown. The remainder of the plasmid sequence, which is circular, is represented as the thin double lines that flank the gp160 gene. The position of the SV40 promoter (SV40 pro.) and poly A addition site (pA) is indicated.

FIG. 2: Linerarized pSV7-ADA-gp160 DNA vector for homologous recombination. After treatment with the restriction enzymes Nde I and Eco NI the linear pSV7-ADA-gp160 DNA, which is largely deleted for gp120 sequence (blue), is gel purified and ready to be used as the ‘vector’ for a homologous recombination reaction. The remainder of the plasmid sequence is represented as the thin double black lines that flank the gp160 gene. The position of the SV40 promoter (SV40 pro.) and poly A addition site (pA) is indicated.

FIG. 3: pCMV-RU570-gp160 mammalian expression vector DNA sequences encoding for HIV strain RU570 gp120 and gp41 and recognition sites for the Hind III, Bsg I and Xho I restriction enzymes are shown. Thin black lines represent the remainder of the circular bacterial plasmid DNA sequence. Positions of the CMV early promoter (CMV pro.) and poly A site (pA) are indicated.

FIG. 4: HRx reaction to insert gp160 or gp120 insert sequences from the RU570 into the pSV7-ADA expression plasmid. The RU570 gp160 insert comprising the gp120 coding sequence and the gp41 DNA coding, was obtained by restriction cleavage of pcDNA3.1-RU570-gp160 with Hind III and Xho I followed by gel purification of the 160 restriction fragment. The RU570 gp120 alone insert was obtained by restriction cleavage of pcDNA3.1-QZND-77-gp160 with Hind III and Bsg 1 followed by gel purification of the gp120 restriction fragment. HRx (represented by black X's) occurs at any point in the sequences that are homologous in the vector and insert sequences. Successful HRx results in the generation of circular plasmids containing RU570-gp120 inserts which can replicate and confer ampicillin resistance to BJ5183 cells.

FIG. 5: Vicriviroc susceptibility assays for RU570-PC (-□-) and RU3570-VCV (-▪-) cultures passaged in PM-1 cells were performed in PBL and dose-response curves representing week 22 (A), week 45 (S), week 47 (C), and week 93 (D) are presented. Curve were generated with PrismGraphPad Software program, Version 4.0 using a non-linear regression 4-parameter logistic curve fit analysis (R² values, RU570-PC: 0.90-0.98, RU570-VCV: 0.80-0.97).

FIG. 6: Schematic of the chimeric envelopes generated by homologous recombination in BJ5183 E. coli bacterial cultures. The hatched bars represent the gp120 sequences from VCVᵣₑₛ gp120 clones and the filled bars represent the envelope sequence from RU570-PC gp160 into which these sequences were recombined. Amino acid changes that were identified in VCVᵣₑₛ gp120 are designated by arrows using HXB2 amino acid coordinates B). HIV-1 pseudoviruses generated with VCVᵣₑₛ chimeric RU570-PC envelopes were analyzed for vicriviroc susceptibility in U87-CD4-CCR5 cells. Dose-response curves for vicriviroc-susceptibility for each HIV-1 pseudovirus (□-RU570-PC gp160, •-RU570-PC-C2-C3ᵣₑₛ, ▪-RU570-PC-C2-V4ᵣₑₛ, ∘-RU570-PC-C2-V5ᵣₑₛ) are represented. Data was analyzed using a non-linear regression 4-parameter logistic curve-fit analysis with PrismGraphPad Software Version 4.0 (R² values, RU570-PC: 0.91, RU570-PC-C2-V5ᵣₑₛ: 0.95).

FIG. 7: The relative infectivity (RLU/p24) of HIV-1 pseudoviruses generated with chimeric envelopes was compared to ADA gp¹⁶⁰ and RU570-PC gp160 pseudoviruses. Data represents the luciferase activity (RLU) in cells 72-hours post-infection that is normalized to the p24 inoculum.

FIG. 8: A) Schematic of the chimeric envelopes generated by homologous recombination in BJ5183 E. coli bacterial cultures. The hatched bars represent the gp120 sequences from VCVᵣₑₛ gp120 clones and the filled bars represent the envelope sequence from ADA gp160 into which these sequences were recombined. Amino acid changes that were identified in VCVᵣₑₛ gp120 are designated by arrows using HXB2 amino acid coordinates. B). Dose-response curves for vicriviroc-susceptibility assays were performed for each HIV-1 pseudovirus (□-ADA, ∘-ADA-V3ᵣₑₛ, ▪-ADA-C2-V5ᵣₑₛ). Data was analyzed using a non-linear regression 4-parameter logistic curve-fit analysis with PrismGraphPad Software Version 4.0 (R² values, ADA: 0.94, ADA-V3ᵣₑₛ: 0.96, ADA-C2-V5ᵣₑₛ: 0.87).

FIG. 9: Vicriviroc dose-response curves for replicating HIV-1 NL4-3-AD8-□, and HIV-NL4-3-AD8 containing the C2-V5ᵣₑₛ-▪- and V3ᵣₑₛ-•-gp120 domains from RU570-VCVᵣₑₛ gp120 in U87-CD4-CCR5 cells.

FIG. 10: Activity of chimeric gp 160 in a pseudotype particle assay. The relative infectivity (RLU/p24) of HIV-1 pseudoviruses generated with RU570-VCVᵣₑₛ/ADA chimeric envelopes at 72 hours after infection. Data represents the fold decrease in luciferase activity (RLU), normalized to p24 inoculum, for pseudoviruses containing chimeric gp160 genes compared with pseudoviruses containing ADA gp 160. The C2-V3, and C2-V5(A) chimeric envelopes produced pseudoparticles sufficiently active for measurement of resistance to the HIV entry inhibitor Vicriviroc, whereas the other chimeras were not sufficiently active or completely inactive.

### 4. DETAILED DESCRIPTION

In accordance with the present invention there may be employed conventional molecular biology, microbiology, and recombinant DNA techniques within the skill of the art. Such techniques are explained in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994).

The invention relates to cloning strategies for Human Immunodeficiency Virus (HIV) genes, in particular HIV envelope genes. This invention is based, in part, on the discovery that bacterial-based homologous recombination (HRx) can be used as a more reliable and efficient method to clone all or part of HIV envelope genes (e.g., all or part of gp 160) into bacterial vectors. In one embodiment, the invention relates to a method of cloning an HIV gene comprising transforming a host cell with a first linearized polynucleotide comprising at least one regulatory element and encoding at least one selectable marker (e.g., antibiotic resistance gene) and optionally, all or part of a polypeptide of interest and a second linearized polynucleotide encoding all or part of a polypeptide of interest, wherein the first polynucleotide and the second polynucleotide each comprise homologous sequences, with respect to each other, sufficient to allow for homologous recombination between the first polynucleotide and the second polynucleotide. In a preferred embodiment, the polypeptide of interest is an HIV envelope protein.

The first polynucleotide of the invention may be a linearized vector comprising at least one regulatory element and encoding a selectable marker. Such regulatory elements are well known in the art and include, but are not limited to, operators, promoters, enhancers, promoter-proximal elements leader sequence, termination codons, polyadenylation signals, replication origins and any other sequences necessary or preferred for the appropriate transcription and subsequent translation of the nucleic acid sequence in the host cell. By way of example, a promoter can be any promoter known to the skilled artisan. For example, the promoter can be a constitutive promoter, a tissue-specific promoter or an inducible promoter. Examples of promoters that may be used in accordance with the invention include, but are not limited to the SV40 early promoter (Bemoist and Chambon, 981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. USA 78:1441-1445), the regulatory sequences of the metallothionein gene (Brinster et al., 1982, Nature 296:39-42), the beta-actin promoter, the CMV promoter, the SR-alpha promoter, the hFer/5V40 promoter, the RSV promoter, the Elf-1 promoter, the Tet promoter, the Ecdyson promoter and a rapamycin promoter. In certain embodiments, it may be desirable to use a constitutive promoter, such as a CMV promoter, beta-actin promoter, SR-alpha promoter or hFer/SV40 promoter.

By way of example, and not limitation, examples of vectors that may be used include, but are not limited to bacterial expression vectors. Examples of bacterial expression vectors include, but is not limited to pBR322, pSV7d, the PUC series of plasmids and pcDNA). Methods for linearizing vectors are know in the art. By way of example, and not limitation the vector may be linearized utilizing restriction enzyme sites in the vector. Also by way of example, and not limitation, PCR amplification may be used to generate the linearized vector.

Selectable markers are known in the art. Examples of selectable markers include, but are not limited to, nucleic acid sequences that encode products that provide resistance against otherwise toxic compounds (e.g., antibiotic); nucleic acid sequences that encode products that are otherwise lacking in the recipient cell (e.g., tRNA genes, auxotrophic markers); nucleic acid sequences that encode products that suppress the activity of a gene product; nucleic acid sequences that encode products that can be identified (e.g., reporter genes, including Beta-galactosidase, fluorescent proteins), nucleic acid sequences that bind products that are detrimental to cell survival and/or function; nucleic acid sequences that inhibit the activity of a gene product (e.g., antisense oligonucleotides or siRNA); nucleic acid sequences that bind products that modify a substrate (e.g. restriction endonucleases); and nucleic acid sequences that can be used to isolate a desired molecule (e.g. specific protein binding sites).

The first polynucleotide may optionally encode all or part of a polypeptide of interest. By way of example, and not limitation. the first polynucleotide can encode all or part of the HIV gp160 envelope protein. Also by way of example, and not limitation, the first polynucleotide can encode all or part of gp120 protein and/or all or part of the gp 41 protein. By way of example, and not limitation, the polynucleotide can encode all or pan of the domains of the gp120 protein including, but not limited to, C1, V1/V2, C2, V3, C3, V4, C4, C5 and V5 or combinations thereof.

The second linearized polynucleotide may encode any polypeptide of interest. In one embodiment the second linearized polynucleotide encodes an HIV protein. Examples of HIV protein of interest include, but are not limited to HIV envelope genes (e.g., 120, gp 160) or parts thereof. Chimeric HIV envelope genes may also be cloned by the instant methods.

By way of example, and not limitation. the second polynucleotide can encode all or part of the HIV gp160 envelope protein. Also by way of example, and not limitation, the second polynucleotide can encode all or part of gp120 protein and/or all or part of the g 41 protein. By way of example, and not limitation, the polynucleotide can encode all or part of the domains of the gp120 protein including, but not limited to, C1, V1/V2, C2, V3, C3, V4, C4, C5 and V5 or combinations thereof.

The second polynucleotide of the invention may be a linearized vector By way of example, and not limitation, examples of vectors that may be used include, but are not limited to bacterial expression vectors. Examples of bacterial expression vectors include, but are not limited to pBR322, pSV7d, the PUC series of plasmids and pcDNA. Methods for linearizing vectors are know in the art. By way of example, and not limitation the vector may be linearized utilizing restriction enzyme sites in the vector. Also by way of example, and not limitation, PCR amplification may be used to generate the linearized vector.

The first polynucleotide and the second polynucleotide each comprise homologous sequences with respect to each other sufficient to allow for homologous recombination between the first polynucleotide and the second polynucleotide. By way of example, and not limitation, the first polynucleotide and the second polynucleotide may each comprise between about 20 to between about 100 nucleotides of homologous sequence. The degree of homology between the homologous sequences of the first polynucleotide and second polynucleotide may be between about 95% to between about 75% homologous (e.g., 95%, 90%, 85%, 80% or 75% homologous).

In a preferred embodiment, the between about 20 nucleotides to between about 100 nucleotides of homologous sequence on each polynucleotide are contiguous nucleotides. By way of example, the first polynucleotide can comprise about 20 nucleotides on each end which are homologous to about 20 nucleotides on each end of the second polynucleotide. (see, e.g., Example 1)

In one embodiment, the first polynucleotide may comprise at least one regulatory element and at least one selectable marker and, optionally encode all or part of an HIV envelope protein and the second linearized polynucleotide may encode all or part of an HIV envelope protein. The first and second polynucleotides may both be linearized vectors. By way of example, and not limitation, the first linearized polynucleotide can comprise the pSV7d ADAgp160 vector restricted with NdeI at position 177 bp in the gp120 coding sequence and with EcoNI at position 1403 in the gp120 coding sequence and contiguous vector sequences containing at least one regulatory element and a least one selectable marker and the second linearized polynucleotide can comprise the entire RU570 gp160 coding sequence generated by restriction enzyme digest or polymerase chain reaction from RU570 expression plasmid, or RU570 HIV viral DNA.

In another embodiment chimeric envelope sequences are produced by the homologous recombination of a first polynucleotide with the second polynucleotide. The chimeric envelope protein produced by the homologous recombination method can comprise HIV envelope sequences derived from at least two strains of HIV. The strains can be from the same lade or different clades (e.g., one strain from Clade B and one starin from Clade G). By way of example and not limitation the first polynucleotide can encode for part of the gp120 protein (e.g., all or part of the C1, V1/V2, C2, V3, C3, V4, V5 and C5 domains or combinations thereof) and/or part of the gp 41 protein or combinations thereof from one strain of HIV and the second polynucleotide can encode can encode for part of the gp120 protein (e.g., all or part of the C1, V1/V2, C2, V3, C3, V4, V5 and C5 domains or combinations thereof) and/or part of the gp 41 protein or combinations thereof from a second HIV strain.

In one embodiment the chimeric envelope sequences produced by the homologous recombination of the first polynucleotide with the second polynucleotide are used to map resistance to an HIV therapeutic (e.g., viral entry inhibitors). The chimeric envelope sequences produced by the homologous recombination method and utilized to map resistance comprise part of an envelope sequence sensitive to the HIV therapeutic and part of an envelope sequence resistant to the HIV therapeutic. To map resistance multiple chimeric envelope sequences can be produced by the homologous recombination methods wherein each chimeric envelope sequence comprises a different portion (e.g., differing lengths of HIV envelope sequences sensitive to the HIV therapeutic and HIV envelope sequences resistant to the HIV therapeutic) HIV envelope sequences sensitive to the HIV therapeutic and HIV envelope sequences resistant to the HIV therapeutic. The resistance determinant is mapped by assaying the chimeric envelope proteins in the presence of a titration of drug (e.g., pseudoparticle assay). By way of example, and not limitation, in the presence of a titration of drug a chimeric envelope protein can be considered resistant if there is a reduction in drug inhibition from 100% (complete inhibition being 100%).

By way of example, and not limitation, the first linearized polynucleotide can comprise the pSV7d ADAgp160 vector restricted with BglII at position 805 bp in the gp160 coding sequence and with BstZ17I at position 907 in the gp120 coding sequence and contiguous vector sequences containing at least one regulatory element and a least one selectable marker and the second linearized polynucleotide comprises the C2-C5 RU570-VCVᵣₑₛ gp120 coding sequence fragment generated by restriction enzyme digest with AleI at position 666 and MfeI at position 1369 in the RU570-VCVᵣₑₛ.

This invention also relates to kits comprising the first polynucleotide and optionally a second polynucleotide as described herein. By way of example, a kit can comprise the linearized first polynucleotide as described herein and a linearized insert fragment as a control.

The host cell may be any cell By way of example, the host cell may be a bacterial cell permissive to recombination. Example include, but are not limited to RecA+ E. Coli cells, BJ5183, KC8, BUN10, V234, MG16J5 and DH 5 alpha. Methods for culturing bacterial cells are known in the art.

This invention also relates to the recombination product produced by the recombination of the first polynucleotide and second polynucleotide. Generally the recombinant product will be in the form of a supercoiled DNA, allowing for ease of amplification and selection in bacterial cells.

### 5. EXAMPLES

### Example 1

### HRx for to Generate HIV gp160 Containing Plasmids

In general, 20 nucleotides of homologous sequence between the vector and insert DNA fragments are sufficient for HRx in bacteria, although longer overlapping sequences can result in more efficient recombination reactions. Recipient vector plasmids for gp160 HRx can be generated by linearizing any existing HIV gp160 expression plasmid. gp160 coding insert sequences for HRx can be generated by restriction enzyme treatment of an existing expression plasmid or HIV molecular clone. In addition, p160 insert sequences can be generated by PCR from HIV molecular clones or from plasma samples from HIV infected patients.

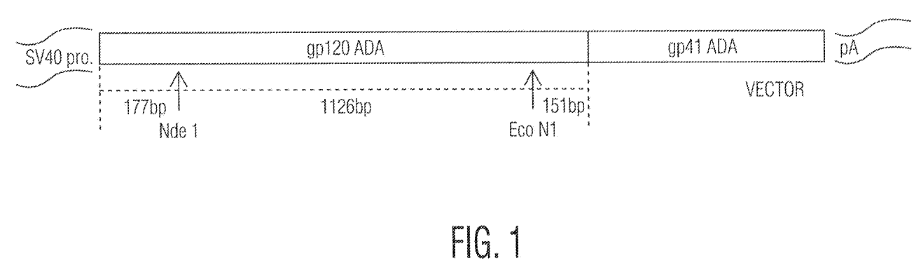

To demonstrate the efficiency of homologous recombination for cloning gp 160, and gp110, an experiment in which linearized gp 60 or gp120 DNA sequences from the HIV RU570 isolate were recombined into the pSV7-ADA-gp160 (FIG. 1A) expression vector was conducted. The pSV7-ADA-gp160 clone contains the gp160 gene from the HIV ADA strain cloned in an expression cassette under the control of the SV40 early promoter, allowing expression in mammalian cells. To prepare pSV7-ADA-gp160 as the vector sequence for HRx full length plasmid DNA was treated with the restriction enzymes Nde I and Eco NI (FIG. 1B) followed by agarose gel isolation of the restricted vector sequence. The Nde I/Eco NI linearized pSV7-ADA-gp160 vector for HRx is deleted for the majority of the gp120 coding sequence, with the exception of the first 177 bp at the 5′ end and the last 151 hp at the 3, end, but the ADA gp4 coding region remains intact. gp 160 DNA sequences remaining in the Nde I/Eco NI linearized pSV7-ADA-gp160 vector are sufficient for HRx reactions to insert full length exogenous gp160 (encoding both gp120 and gp 41) or exogenous gp120 sequence alone.

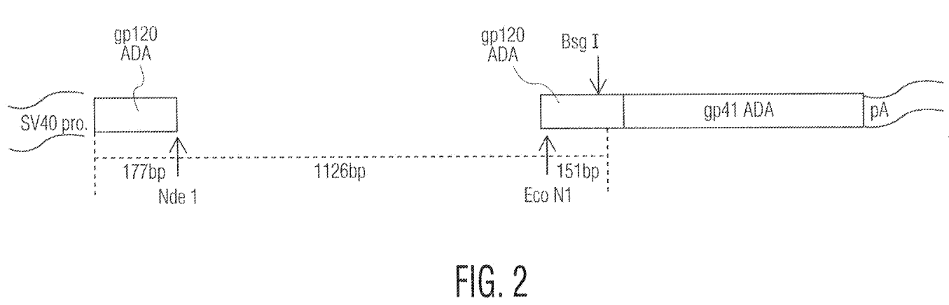

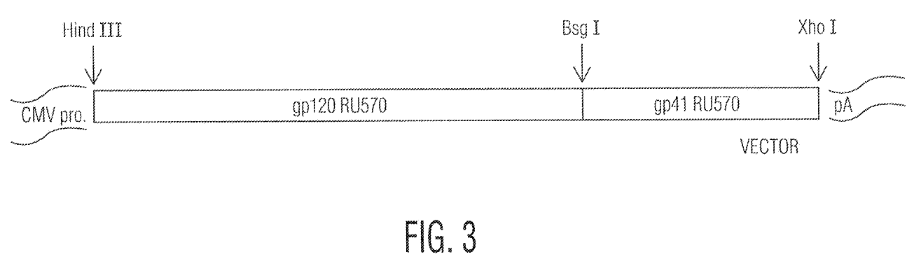

Insert fragments for recombination of the full-length gp160 gene, or gp120 DNA sequence alone, into the linearized pSV7-ADA-gp160 vector were prepared from the pcDNA3.1-RU570-gp160 expression vector (FIG. 2a). The full-length RU570 gp160 DNA insert sequence was obtained by digestion of pcDNA3.1-RU570-gp160 with the restriction enzymes Hind III and Xho I followed by agarose gel purification of the 2600 bp fragment. The gp120 DNA insert sequence was obtained by cleavage of pcDNA3.1-RU570-gp160 with Hind III and Bsg I to obtain a 1500 bp fragment which was also isolated by gel purification.

HRx reactions were carried out in the recA⁺ E. coli strain BJ5183. For HRx the pSV7-ADA-gp 160 vector and RU570 gp160 or gp120 insert DNA fragments were nixed with a 5 molar excess of the insert fragment and then transformed into competent BJ5183 cells using standard procedures (FIG. 2). For a control BJ5183 cells were also transformed with linearized pSV7-ADA-gp160 vector DNA. After transformation the bacterial cells were plated on culture plates containing carbenicillin (100 μg/ml) and incubated at 30° C. After 24 hour incubation the number of colonies on the culture plates was scored (see Table 1). Colonies from each plate were then propagated overnight at 30° C. in liquid culture and tested by PCR analysis for the presence of full length gp160 or gp120 (Table 1).

The results of the gp 160 and gp120 HRx experiment demonstrate that gp160 and gp120 can be cloned is this system with a high success rate. For gp160 HRx 69% of the screened bacteria colonies contained full-length gp160. This compares favorably with results from conventional ligation cloning of gp160 DNA sequences in which full-length gp160 sequences are rarely identified in more than 4% of colonies and often no positive clones are obtained. For gp120 cloning by HRx 100% of the tested colonies contained full length gp120. Conventional ligation cloning of gp120 usually results in a success rate of 10-20%. These results demonstrate that the HRx system for cloning of HIV gp160 and gp120 sequences is efficient with a higher success rate as compared to conventional ligation cloning.

TABLE 1
Efficiency of HRx of RU570 gp160 or gp120 Coding Sequence
were Inserted into the pSV7-ADA Vector

| HRx | # OF | TOTAL | POSI- | NEGA- | |
|---|---|---|---|---|---|
| REACTION | COLONIES | TESTED | TIVE | TIVE | PERCENT |
| RU570 | 50 | 26 | 18 | 8 | 69 |
| (gp160) | | | | | |
| RU570 | >100 | 24 | 24 | 0 | 100 |
| (gp120) | | | | | |
| Control | 1 | 1 | 0 | 1 | 0 |

### Example 2

### Material and Methods

### Reagents

Vicriviroc was synthesized at Schering-Plough Research Institute, Kenilworth, N.J. (Strizki et al (2005) Antimicrobial Agents and Chem. Vol. 49 912) page 4911) The pSI expression vector was purchased from Promega Corp. (Madison, Wis.).

### HIV-1 Plasmids and HIV-1 Primary Isolates

The HIV-1 clade G RU570 primary isolate and the pNL4-3-AD8 molecular clone were obtained from the NIH AIDS Research and Reference Reagent program. The pNL4-3E⁻Luc⁺ and pSV7d-ADA gp160 plasmids were obtained from John Moore, Weill Medical College of Cornell University, New York, N.Y. The pcDNA3.1-RU570 gp160 clone (Parental) was constructed as follows. PBL cultures were infected with the RU570 primary isolate and an HIV DNA fragment corresponding to HXB2 nucleotide sequence 5739→8920 was PCR amplified from cell DNA using Platinum Pfx DNA polymerase (InVitrogen Corp., Carlsbad, Calif.) with the following primers:

Vpr-(F)-5′-ATAAGAATTCTGCAACAACTGCTG-3′
(HXB2: 5739-5762)
Nef-(R)-5′-CTCCATGTTTTTCTAGGTCTCGAGA-3′
(HXB2: 8896-8920)

The PCR product was gel purified and cloned into pCR®-Blunt II Topo vector (Invitrogen Corp.). RU570 gp160 was amplified from this construct using the following envelope specific primers:

| | |
|---|---|
| Forward: | 5′CACCATGAGAGTGAAGGGGATACAGAAG-3′ |
| Reverse: | 5′CCTCCCATTTTATAGCAAAGCTCTTTC-3′ |

The 2.5 kb envelope PCR product was gel purified and cloned directly into pcDNA3.1, Version C vector (Invitrogen Corp).

### Cell Lines

The neoplastic T-cell line, PM-1 was obtained from the NIH AIDS Research and Reference Reagent program. PM-1 cells were maintained in RPMI medium supplemented with 10% fetal bovine serum (FBS). U87 astroglioma cells expressing CD4 only, or with CCR1, CCR2, CCR3, CCR5 or CXCR4 were obtained from Dr. Dan Littman, New York University. Cells expressing CD4 only were maintained in DMEM supplemented with 10% FBS and 500 μg/ml G418 and cells expressing both CD4 and chemokine receptors were maintained in the same medium plus 1 μg/ml puromycin. 293T cells (CRL-11268) were purchased from ATCC (Manassas, Va.) and maintained in DMEM supplemented with 10% FBS and 300 μg/ml G418. PBL preparations from normal blood donors were obtained by leukophoresis performed at the New York Blood Center. PBLs from each donor were purified by centrifugation over a Ficoll-Hypaque density gradient (2000 rpm, 30 min). The cells were washed twice with PBS and pools from 6-8 donors were frozen in aliquots (5×10⁷ cells per vial). Cells were cultured in RPMI supplemented with 10% FBS, 50 U/ml interleukin-2 (IL-2) and stimulated with 5 μg/ml phytohemagglutinin for 3 days prior to infection.

### In-Vitro Generation of RU570 Virus Cultures Resistant to Vicriviroc

The Hut 78 derived PM-1 (Lusso, P., et al (1995). J Virol 69(6), 3712-20) T-cell line was infected with a parental PBL stock of HIV-1 RU570 for one week prior to the initiation of vicriviroc selection starting at 0.1 nM. PM-1 cell cultures were passaged weekly by transferring 1 ml of cell culture supernatant and one tenth of the infected cells into fresh PM-1 cultures (1-2×10⁶) in the absence (passage control culture, RU570-PC) or presence (RU570-VCV) of vicriviroc. Culture supernatants were subsequently used to re-infect fresh PM-1 cells weekly. Viral supernatants and infected cells were collected periodically for p24 antigen titers, vicriviroc sensitivity testing in PBL described below in the viral replication assay section, and sequence analysis. RU570 virus initially grew poorly in the presence of vicriviroc, hence the concentration of vicriviroc was held below 1 nM for the first 12 weeks. As the replication of RU570 in the presence of vicriviroc increased, the concentration of vicriviroc was slowly increased to 400 nM between weeks 12 and 22 and this concentration was held constant until week 36. To select for minor populations of highly resistant variants that may have emerged, the concentration of vicriviroc was escalated to 10 μM at week 36 and cultures were maintained in 10 μM vicriviroc until viral replication remained consistent. By approximately week 56, VCV-treated cultures were replicating to similar levels compared to the passage control cultures and maintained consistently robust growth up to 93 weeks when cultures were terminated.

### Viral Replication Assays

IL-2/PHA-stimulated PBL were seeded into 96-well plates (200,000 cells/well) pretreated with an equal volume of medium plus vicriviroc or medium only at 37° C. for 1 hour. The plates were centrifuged at 300 g for 10 min and the media aspirated and replaced with 20 μl of fresh medium with or without compound. The cells were then infected in triplicate using 20 μl of viral supernatant (RU570) from 3 hr to overnight at 37° C. The cells were washed twice with PBS to remove residual viral inoculums and cultured in the presence or absence of vicriviroc for 4-6 days. Viral replication was assessed by measuring p24 antigen production by ELISA. The IC₅₀ and IC₉₀ values for vicriviroc susceptibility with these viruses was determined by analyzing dose-response curves using a 4-parameter logistic curve fit model with Prism GraphPad Software Version 4.0 (San Diego, Calif.).

Genotypic Analysis of HIV-1 gp120 Sequences

U87-CD4-CCR5 cells were infected with RU570 passage control or RU570 vicriviroc-resistant viruses collected following 93 weeks in culture. 48 hrs post infection total cellular DNA was isolated using Qiagen DNeasy tissue kit (Valencia, Calif.) and HIV-1 gp120 sequences were amplified by DNA-PCR from genomic DNA using the following restriction-site designed primers: SfiI-gp120 1F 5′-GCGGCCCAGCCGGCCAVAGTGAWGGRGAWACAGARGAATTGG-3′ corresponding to the HXB2 genome nucleotide sequence 6228-6255 and XhoI-gp120 1R 5′-GGCTCGAGATCTTTTTTCTCTCYSCACCACTCTTCTCY-3′ corresponding to the HXB2 genome nucleotide sequence 7729-7757. Special nucleotide designators are as follows: V=A/C/G, W=A/T, R=A/G, Y=C/T. HIV-1 gp120 sequences were amplified with Roche Expand High Fidelity PCR system (Roche Applied Science, Mannheim, Germany) using the following 30 cycle elongation program: 94° C., 30 sec., 55° C., 45 sec., and 68° C., 4 min. PCR products were digested with SfiI and XhoI restriction endonucleases (New England Biolabs, Beverly, Mass.), gel purified on 1% agarose and cloned directly into pSECTag2 (Hygro A) vector (InVitrogen Corp., Carlsbad, Calif.). DNA sequence analysis of individual clones was performed using a CEQ 2000 Dye terminator cycle sequencer (Beckman-Coulter Inc., Fullerton, Calif.).

Homologous Recombination of HIV-1 gp120 Fragments into pSV7d-ADAgp160 and pSI-RU570 PC gp160 Expression Vectors

The pSI-RU570 PC gp160 expression vector was constructed following PCR amplification of gp160 using restriction-site designed primers: MluI-Env4F 5′-GGGACGCGTATGAVAGTGAWGGRGAW-3′ and XbaI-Env4R 5′-AAATCTAGGTTTGACMAYTTTGCCHCCCATYTTA-3′ corresponding to envelope sequences flanking the start and stop codons of HIV-1 gp160, respectively. Special nucleotide designators are M=A/C and H=A/C/T. HIV-1 gp160 sequences were amplified with Roche Expand High Fidelity PCR system (Roche Applied Science, Mannheim, Germany) using the following 30 cycle elongation program: 94° C., 30 sec., 55° C., 45 sec., and 68° C., 7 min. PCR products were digested with MluI and XbaI restriction endonucleases (New England Biolabs, Beverly, Mass.), gel purified on 1% agarose and cloned directly into the pSI expression vector.

Homologous recombination of RU570 vicriviroc-resistant gp120 (RU570-VCVᵣₑₛ) into the pSI-RU570 passage control gp160 (RU570-PC) expression vector was performed as follows. The RU570-VCVᵣₑₛ gp120 clone #8 in pSECTag2 was digested with AleI and MfeI restriction endonucleases and the 730-bp C2-C5 gp120 fragment (HXB2 gp120 nucleotides 681-1432; gp120 at position 228-478) was gel-purified on 1.0% agarose. The pSI-RU570-PC expression vector was digested with BsrGI and SbfI restriction endonucleases, followed by alkaline phosphatase treatment. The 5.8 kb vector fragment was gel purified on 1% agarose. Homologous recombination was performed using a 10-fold molar excess of gp120 fragment to vector for transformation of chemically-competent BJ5183 E. coli, and individual colonies were screened by PCR for successful recombination.

Homologous recombination of the V2-C5 gp120 fragment from RU570-PC gp120 clone #16 and RU570-VCVᵣₑₛ gp120 clone #8 was performed following restriction endonuclease digestion of constructs with MfeI and gel purification of the 838 bp V2-C5 gp120 fragments (HXB2 gp120 nucleotides 533-1431; gp120 at position 185-478). The pSV7d-ADA gp160 expression vector was digested with BglII and BstZ17I restriction endonucleases, alkaline phosphatase treated and the 5.1 kb vector was gel purified on 1% agarose. A 50:1 ratio of V2-C5ᵣₑₛ gp120 fragment to vector was used to transform chemically-competent BJ5183 E. coli, and individual colonies were screened by PCR for successful recombination.

### Generation and Characterization of HIV-1 Pseudoviruses

HIV-1 pseudoviruses were produced in 293T cells by calcium phosphate transfection of pNL4-3E⁻Luc⁺ and HIV-1 envelope expression vectors using ProFection® Mammalian Transfection system (Promega Corp., Madison, Wis.). HIV-1 pseudovirus was harvested in culture supernatants 48 hrs post-transfection and supernatants were clarified of cell debris by centrifugation at 1500 g for 10 min. Single-cycle infection assays were generally performed on the same day HIV-1 pseudovirus was harvested. To assess susceptibility to CCR5 co-receptor antagonists, 5,000 U87-CD4-CCR5 cells/well were seeded into 96-well luminometer plates (Perkin Elmer) and plates were incubated overnight at 37° C. The next day serial 10-fold dilutions of inhibitor in cell culture medium (10 μM→1.0 μM) were added to wells one hour prior to the addition of HIV-1 pseudovirus plus inhibitor. Plates were incubated for 72 hours, and luciferase activity was analyzed by adding 50 μl of BrightGlo™ luciferase assay buffer and plates were read on a Dynex luminometer (300 mSec/well). Relative light units (RLU) were normalized to virus dose, measured as ng p24, and percent inhibition, was calculated as follows: 100−[average normalized RLU for HIV-1 pseudovirus plus drug/average normalized RLU for HIV-1 pseudovirus from control wells without drug]×100. Dose-response data was analyzed using a non-linear regression 4-parameter logistic curve fit program with Prism GraphPad Software Version 4.0 (San Diego, Calif.).

### HIV-1 Molecular Clones

The pNL4-3-AD8 molecular clone was modified by deleting 650 bp of cDNA sequence flanking the 5′LTR, thereby creating a unique Stu I site within gp120. The ADA-V3ᵣₑₛ and ADA-C2-V5ᵣₑₛ chimeric envelopes were digested with StuI and AleI restriction endonucleases and the 1.2 kb env fragments corresponding to at 203-608 (HXB2 gp160 aa coordinates) were gel purified on 1% agarose. These fragments were cloned directly into pNL4-3-AD8 digested with the same restriction endonucleases and all constructs were verified by DNA sequence analysis.

### Generation of Replication-Competent HIV-1 Viruses

293T cells were transfected with pNL4-3-AD8, pNL4-3-AD8-C2-V5ᵣₑₛ, and pNL4-3-AD8-V3ᵣₑₛ molecular clones using Superfect transfection reagent (Qiagen Corp., Vencia, Calif.) and HIV-1 virus was harvested in supernatants 48 hr post transfection.

### Nucleotide Sequence Accession Numbers

The following HIV-1 envelope sequences have been submitted to GenBank: 2 sequences for RU570 gp160 (Accession no. EU090200-EU090201) and 22 gp120 sequences for RU570-derived viruses (Accession no. EU090202-EU090223).

### Results

### Generation of Vicriviroc-Resistant RUC570 HIV-1 Variant in PM-1 Cells

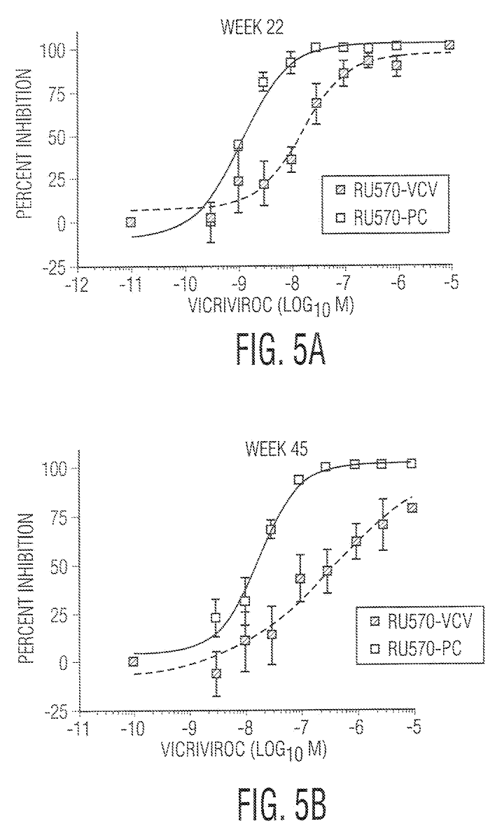

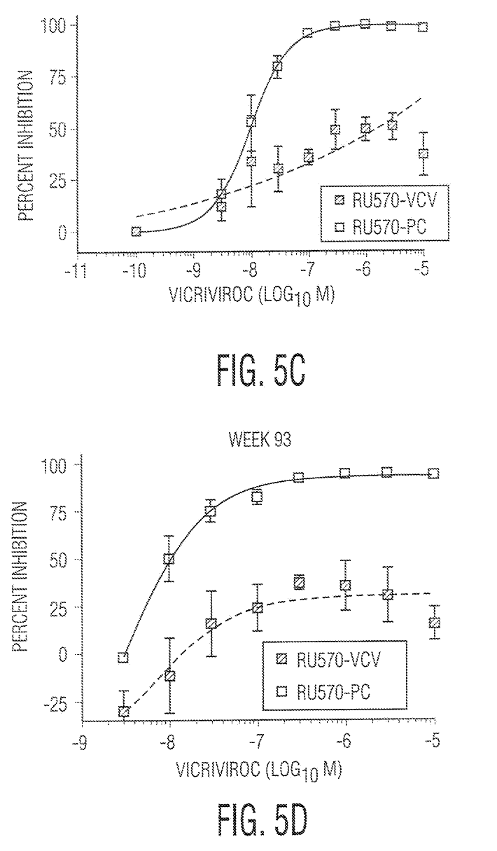

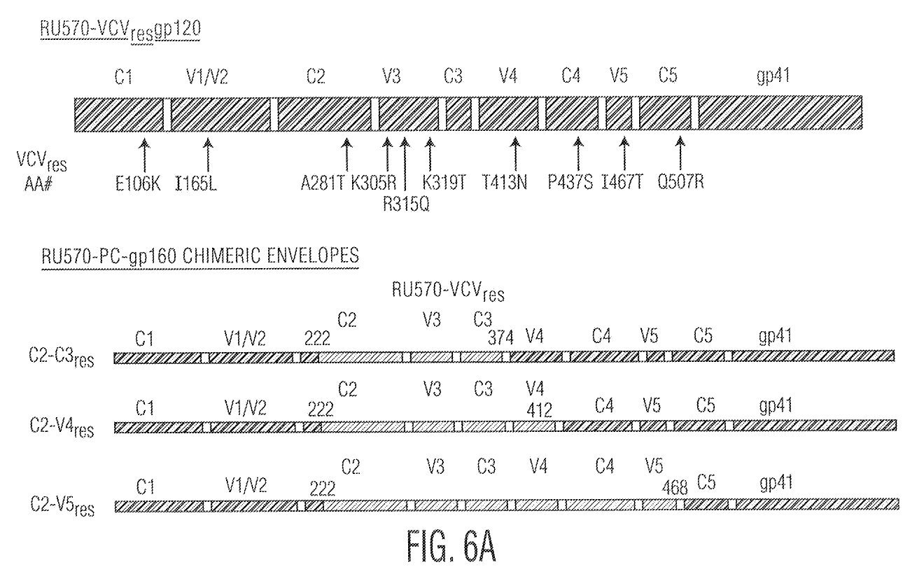

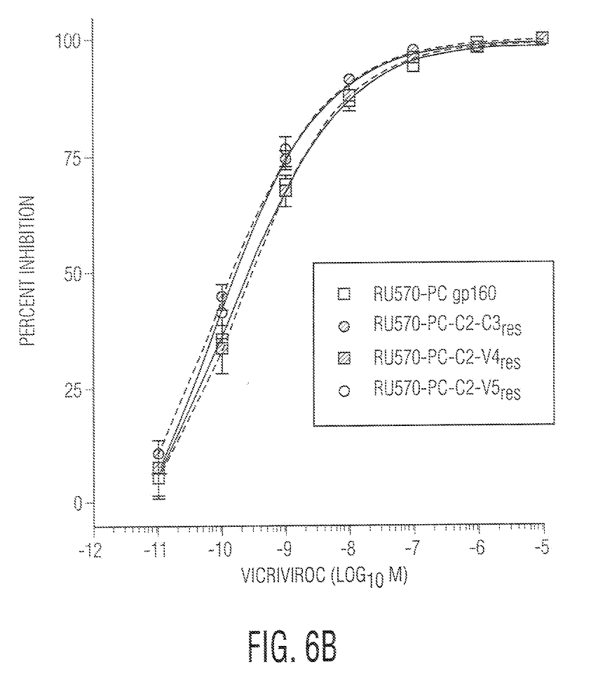

PM-1 viral cultures were established as described in the materials and methods section and were assessed periodically for susceptibility to vicriviroc in PBL replication assays between weeks 12-93. Vicriviroc dose-response curves for viruses obtained from weeks 22 (FIG. 5A), 45 (FIG. 5B), 47 (FIG. 5C), and 93 (FIG. 5D) are depicted in FIG. 1. The RU570-VCV virus was completely susceptible to vicriviroc until week 45 showing IC₅₀ values <50 nM and maximum percent inhibition equivalent to 100% in dose response curves similar to that depicted at week 22 (FIG. 6A). At weeks 45 and 47 incomplete dose response curves to vicriviroc were observed (FIGS. 6B and 6C) with maximal percent inhibition approximately 76 and 45 percent, respectively. As expected the RU570 passage control virus remained susceptible to vicriviroc through 93 weeks of cell culture. By week 93 complete resistance in the vicriviroc treated culture was demonstrated by a flat dose-response curve to drug and a maximum percent inhibition at approximately 30 percent (FIG. 6D).

In order to determine that viral replication in the presence of vicriviroc was not due to co-receptor switching of this primary isolate, we infected U87-CD4 cell lines that express CXCR4 with virus from the week 93 cultures. As has been reported for other in-vitro generated HIV-1 resistant variants to CCR5 co-receptor antagonists (Kuhmann, S. E., et al (2004). J Virol 78(6), 2790-807; Trkola, A., et al (2002). Proc Natl Acad Sci USA 99(1), 395-400; Westby, M., et al (2007). J Virol 81(5), 2359-71), the RU570 vicriviroc-resistant variant (RU570-VCVᵣₑₛ) from week 93 did not replicate in U87-CD4-CXCR4 cells demonstrating that this resistant variant remained CCR5-tropic only (data not shown).

Clonal Sequence Analysis of HIV-1 gp120.

U87-CD4-CCR5 cells were infected with week 93 cultures for RU570-PC and RU570-VCVᵣₑₛ virus in the absence and presence of vicriviroc, respectively. HIV-1 gp120 sequences were amplified by PCR from genomic DNA isolated from infected cells 48 hrs post-infection and clonal sequence analysis of gp120 was performed. The amino acid changes in gp120 identified between the RU570-PC and RU570-VCVᵣₑₛ included ten dominant amino acid changes (present in ≧50% of resistant clones) that were identified, throughout the RU570-VCVᵣₑₛ gp160 protein sequence, following analysis of 18 clones compared with the amino acid sequence of the RU570-PC virus (5 clones). The following dominant amino acid changes were identified: E106K in C1, I165L in V2, A281T in C2, K305R, R315Q, and K319T in the V-3 loop, T413N in V4, P437S in C4, I467T in V5, and Q507R/E in C5 (see FIG. 2). In addition, two amino acid changes in C3 (S363P and T373A), two additional changes in the V3 loop (I317F and G321D) and one amino acid change in C5 (K503R) were found in <50 percent of the clones.

### Generation of Chimeric Envelopes by Homologous Recombination in Bacteria.

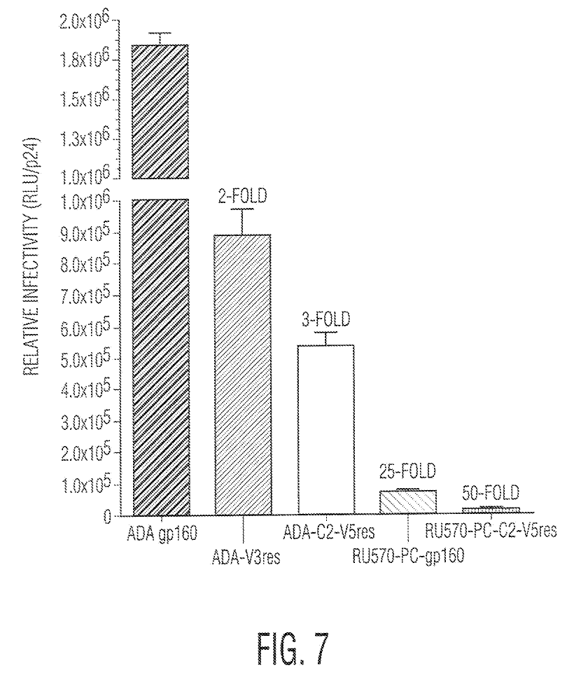

Single-cycle assays using pseudotyped HIV-1 particles that express a reporter gene in infected cells are used to address the susceptibility of HIV-1 envelope clones to co-receptor antagonists. A luciferase reporter-gene located in the nef-coding region and a frame-shift mutation in the envelope gene in pNL4-3-Env⁻Luc⁺ limits the detection of pseudovirus infectivity to a single-round. These assays are also being used to diagnose co-receptor usage for patients entering clinical trials for CCR5 antagonists (Coakley, E., et al (2005). Curr Opin Infect his 18(1), 9-15). Unfortunately, efforts to generate pseudoviruses with gp 160 clones obtained from RU570-VCVᵣₑₛ cultures using the standard pNL4-3Env⁻Luc⁺ HIV-1 replication-incompetent vector resulted in pseudovirus stocks with no detectable luciferase activity. In addition, a majority of the gp110 clones from RU570 passage control (PC) virus failed to generate viable pseudotyped HIV-1 particles. Following screening of numerous gp160 clones we eventually identified a single functional gp160 clone that could be pseudotyped with the HIV-1 vector (FIG. 7)

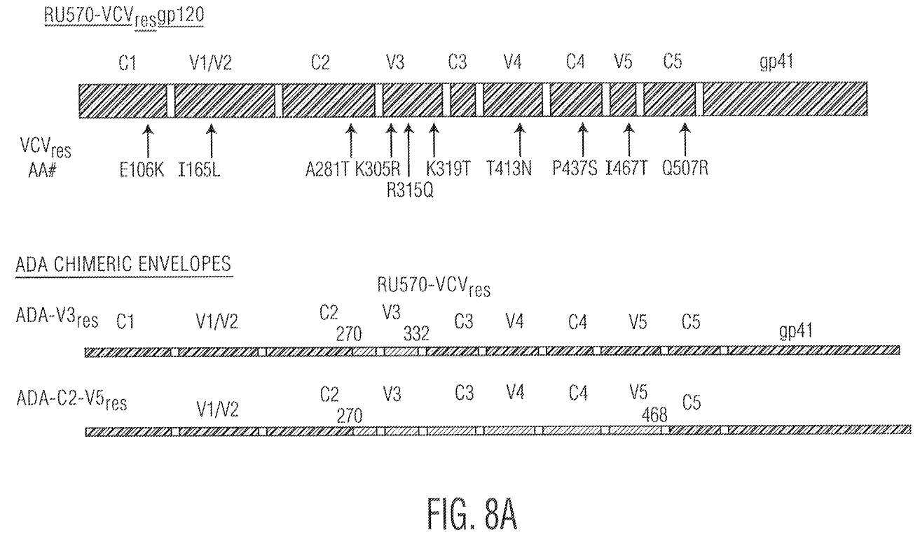

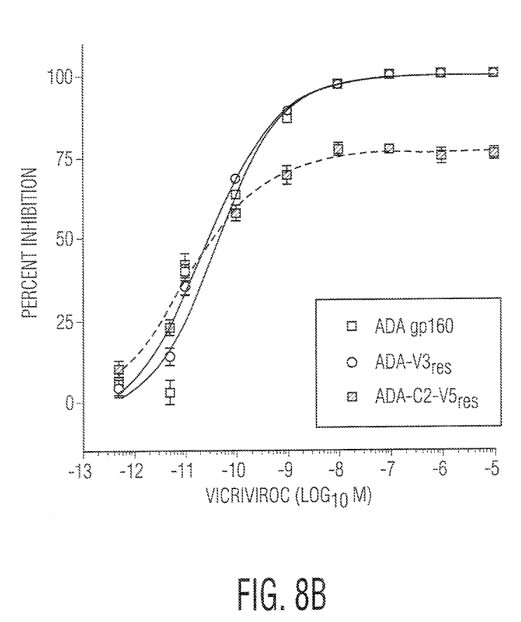

Therefore, we used an alternate strategy to generate functional envelope clones from resistant viruses. Making use of homologous recombination in bacteria, we cloned different gp120 domains from the RU570-VCVᵣₑₛ clones into the background of the RU570-PC gp160 backbone in order to determine if domain swapping would result in the transfer of the resistance phenotype into this vicriviroc-susceptible passage control envelope. Based on numerous studies using restriction-site fragment swapping for mapping co-receptor determinants within gp120 within the same HIV strain (Cho, M. W et al (1998) J Virol 72(3); 21509-15; Choe, H., et al (1996). Cell 85(7), 1135-48; Cocchi, F., et al (1996). Nat Med 2(11), 1244-7), we reasoned that resistance might also tract with domain swapping. Following homologous recombination of VCVᵣₑₛ gp120 into the RU570-PC gp 160 construct, the chimeric envelope clones were sequenced to determine the co-ordinates of recombination and were then used to generate HIV-1 pseudotyped particles. FIG. 6A depicts the 3 homologous recombinant envelopes that were obtained in the background of RU570-PC gp160. Vicriviroc dose-response experiments in U87-CD4-CCR5 cells were performed using HIV-1 particles pseudotyped with the chimeric envelopes. As expected the control RU 570-PC envelope was completely susceptible to vicriviroc with an IC₅₀ value of 0.1 nM and a 100% maximum inhibition. Surprisingly none of the chimeric envelopes containing varying lengths of C2-V5 VCV, gp120 were resistant to vicriviroc. These chimeric pseudotyped HIV-1 particles displayed maximum inhibition levels of 100% at concentrations of vicriviroc ≧100 nM and the IC₅₀ values calculated from dose-response curves depicted in FIG. 2B ranged between 0.07-0.2 nM. Since we routinely analyze the infectivity of pseudoviruses by comparing the ratio of the luciferase activity (RLU) produced per ng of p24 inoculum for each virus (relative infectivity), we found that the RU570-PC and chimeric envelopes had significantly reduced relative infectivity (25-fold to 50-fold lower) in comparison with HIV-1 particles pseudotyped with ADA envelope (FIG. 7). Since the pseudotyped ADA envelope produced such robust activity, we tried homologous recombination of VCVᵣₑₛ gp120 with this completely heterologous, vicriviroc susceptible envelope. Two recombinants containing different fragment lengths of RU570-VCVᵣₑₛ gp120 in ADA gp160 (ADA-V3ᵣₑₛ; HXB2 coordinates: aa 270-332; ADA-C2-V5ᵣₑₛ; HXB2 coordinates: aa 270-468) were generated (FIG. 8A) and characterized for infectivity and susceptibility to vicriviroc in single-cycle HIV-1 pseudovirus assays.

Characterization of HIV-1 Pseudoviruses Containing ADA Chimeric Envelopes

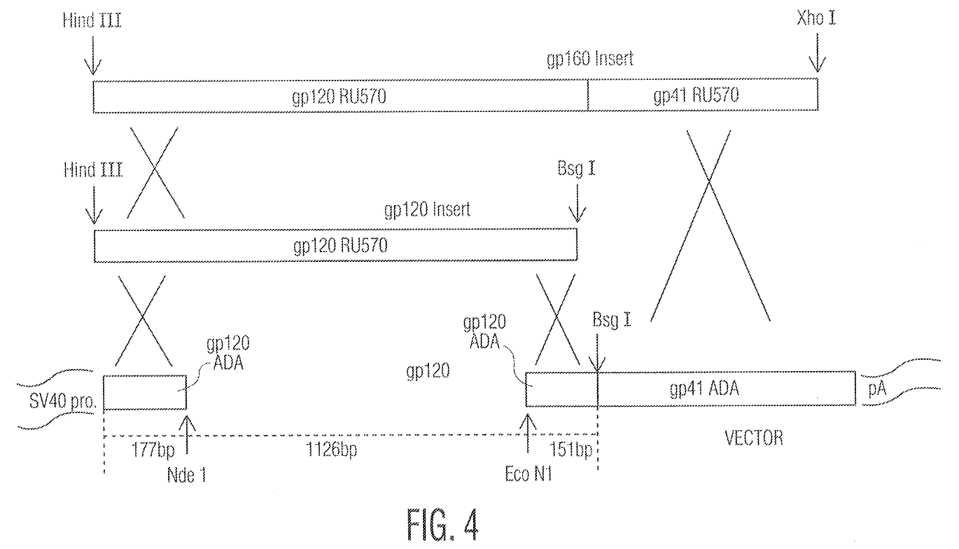

The luciferase activity for pseudoviruses generated with the ADA chimeric envelopes was enhanced significantly relative to the RU570-PC chimera although it was about 2 to 3-fold lower than wt ADA envelope (FIG. 7). We next performed vicriviroc susceptibility assays using the pseudoviruses generated with the ADA chimeric envelopes. Assays were conducted in U87-CD4-CCR5 cells using serial 10-fold dilutions of vicriviroc spanning a 7-8 log₁₀ molar range in inhibitor concentration and vicriviroc susceptibility curves for these pseudoviruses are shown in FIG. 4B. As expected, control HIV-1 particles pseudotyped with ADA gp160 were completely susceptible to vicriviroc with an IC₅₀ value of 0.04 nM and a maximum response of 100 percent inhibition. Interestingly, pseudoviruses generated with the ADA-V3ᵣₑₛ chimeric envelope containing only the V-3 loop region from RU570-VCVᵣₑₛ gp120 were completely susceptible to vicriviroc despite containing 3 dominant mutations in V-3; whereas pseudoviruses containing the ADA-C2-V5ᵣₑₛ chimeric envelope exhibited a resistant phenotype showing a reduction in the maximum inhibition (MI) in vicriviroc dose-response curves. This resistant phenotype has been previously reported for other in-vitro generated resistant variants (Pugach, P., et al (2007). Virology 361(1), 212-28; Westby, M., et al (2007). J Virol 81(5), 2359-71). This data suggests that the additional C3-V4-C4-V5 domains of gp120 are necessary for maintaining vicriviroc resistance in the background of ADA gp160. Since the V3-loop region from RU570-VCVᵣₑₛ gp120 within a heterologous background did not recapitulate resistance, these V3-loop changes are most likely context dependent.

### Effect of Viral Inoculum on Maximum Inhibition Values

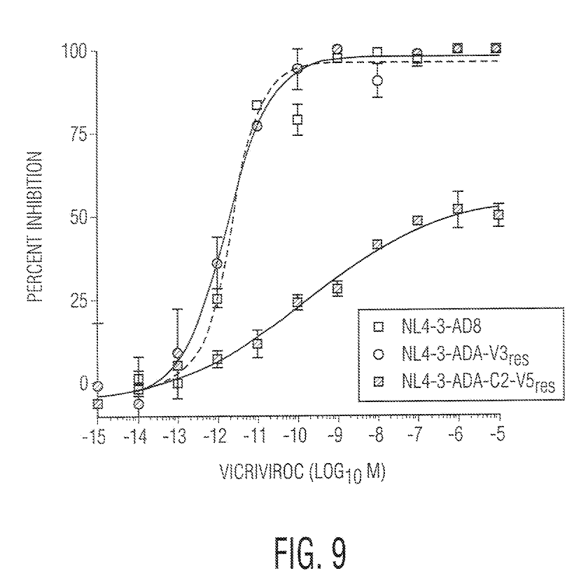

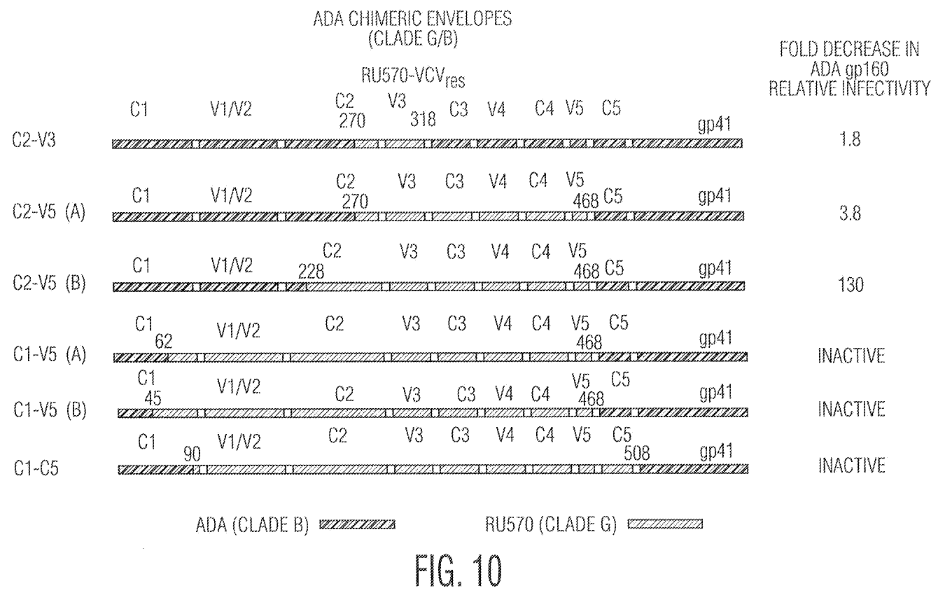

Since the ADA-C2-V5ᵣₑₛ chimeric envelope displayed a vicriviroc-resistance phenotype and the activity was 25-fold higher compared to the RU570-PC-C2-V5ᵣₑₛ chimeric, we tested increasing doses of virus inoculum with the RU570 chimeric to see if this enabled the detection of resistance for this envelope. Because of the limited infectivity of this chimeric envelope (FIG. 9), we were only able to examine a I O-fold range in p24 inocula. Even with very high amounts of inoculum (90 ng p24) no change in the 100% maximum inhibition occurred. Intriguingly, when the ADA-C2-V5ᵣₑₛ pseudovirus infections were examined using increasing amounts of virus inocula the MI for vicriviroc decreased as the level of virus increased (FIG. 9). At high virus inocula (70 ng p24) the plateau level was approximately 55%, however the plateau level increased with decreasing concentrations of virus to approximately 80% MI (2 ng p24). This effect was not seen for HIV-1 pseudoviruses generated with the parental ADA gp160 or RU570-PC 160 constructs. Using a similar range in virus inocula the MI values for these viruses remained at 100% regardless of the virus concentration (data not shown).

Vicriviroc Susceptibility Assays with Replication Competent Chimeric HIV-1 Viruses

CCR5 inhibitor resistance was previously reported to be influenced by the assay format in which resistance is evaluated (Pugach, P., et al (2007). Virology 361(1), 212-28). In assays performed in PBMCs, it was noted that differences were found in the phenotype of a resistant virus displaying non-competitive resistance in a single-cycle format (plateau effect) versus replication enhancement in a multi-cycle assay. In order to evaluate differences in susceptibility profiles between single-cycle and multi-cycle infection assays in U87-CD4-CCR5 cells, we generated replication competent viruses expressing the chimeric gp120 envelopes. Restriction fragments corresponding to HXB2 aa 203-608 within ADA-V3ᵣₑₛ and ADA-C2-V5ᵣₑₛ chimeric envelopes were transferred to an NL4-3-AD8 HIV-1 molecular clone by unique restriction-site digestion and relegation. Infectious virus generated in 293T cells was used to infect U87-CD4-CCR5 cells in the presence of increasing concentrations of vicriviroc. Dose-response data for these viruses is represented in FIG. 9. The replication competent HIV-1 virus containing the ADA-C2-V5ᵣₑₛ chimeric envelope sequence was resistant to vicriviroc as demonstrated by the incomplete dose-response curve to vicriviroc displaying a MI at approximately 50 percent in U87-CD4-CCR5 cells. This resistant virus appears to exhibit the same resistance phenotype (plateau effect) in both assay formats. Since the previously published results were performed in PBMCs which would contain secreted chemokines, our results are not directly comparable to those of Pugach et al. because of the differences in experimental conditions. Both NL4-3-AD8 and the NL4-3-AD8-V3ᵣₑₛ virus containing just the V3-loop region from the resistant gp120 clone were completely susceptible to vicriviroc (IC₅₀=0.025 nM).

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Patents, patent applications, publications, product descriptions, and protocols are cited throughout this application, the disclosures of which are incorporated herein by reference in their entireties.

1. A method of cloning an HIV gene comprising transforming a host cell with a first linearized polynucleotide comprising at least one regulatory element and encoding at least one selectable marker and a second linearized polynucleotide encoding a polypeptide of interest, wherein the first polynucleotide and the second polynucleotide each comprise homologous sequences sufficient to allow for homologous recombination between the first polynucleotide and the second polynucleotide.

2. The method of claim 1, wherein the first polynucleotide and the second polynucleotide comprise between about 20 to between about 100 nucleotides of homologous sequence.

3. The method of claim 1, wherein the degree of homology between the homologous sequences of the first polynucleotide and second polynucleotide is between about 95% to between about 75% homologous.

4. The method of claim 1, wherein the first linearized polynucleotide is a vector and the second linearized polynucleotide is a vector.

5. The method of claim 4, wherein the vector is a bacterial expression vector selected from the group consisting of pBR322, pSV7d, the PUC series of plasmids and pcDNA.

6. The method of claim 1, wherein the first polynucleotide further encodes all or part of the polypeptide of interest.

7. The method of claim 1, wherein the HIV gene is the gp 160 envelope protein gene.

8. The method of claim 3, wherein the second linearized polynucleotide encodes all or part of the gp 160 envelope protein.

9. A host cell comprising the linearized polynucleotides of claim 1.

10. The host cell of claim 9, wherein the host cell is bacterial cell permissive to recombination.

11. The bacterial cell of claim 10, wherein the cell is selected from the group consisting of RecA+E. Coli cells, BJ5183, KC8, BUN10, V234, MG16J5 and DH 5 alpha.

12. The polynucleotide sequence which is the product of the homologous recombination of claim 1.

13. The polynucleotide of claim 12, wherein the polynucleotide is a supercoiled DNA.
